(19) United States
(12) Patent Application Publication
(10) Pub. No.: US 2010/0291264 A1
(43) Pub. Date: Nov. 18, 2010

(54) NOVEL STRAIN IN NATTO-PRODUCING BACTERIA AND SOFT NATTO PRODUCED BY USING SAID STRAIN

(75) Inventor: Hiroshi Takemura, Aichi (JP)

Correspondence Address: OBLON, SPIVAK, MCCLELLAND MAIER & NEUSTADT, L.L.P., 1940 DUKE STREET, ALEXANDRIA, VA, 22314, US

(73) Assignees: MIZKAN GROUP CORPORATION, Handa-shi, Aichi (JP); MIZKAN CO., LTD, Handa-shi, Aichi (JP)

(21) Appl. No.: 12/596,990

(22) PCT Filed: Apr. 21, 2008
(86) PCT No.: PCT/JP2008/057661

§ 371 Date: Oct. 22, 2009

(30) Foreign Application Priority Data
Apr. 25, 2007 (JP) 2007-115110

(51) Int. Cl. A23L 1/20; C12N 1/20

(52) U.S. Cl. 426/46; 426/634; 435/252.5

(57) ABSTRACT

[Object] To provide a strain in natto-producing bacteria that is suitable for the production of soft natto (a fermented soybean product).

[Means for Solving the Problems] A strain in natto-producing bacteria that is suitable for the production of soft natto is provided by isolating natto-producing strains from chungkookjang, which is a fermented food having been taken in Korea, and selecting an appropriate natto-producing strain using the stickiness and hardness of natto as indications. Different from the existing natto-producing bacteria, this strain is characterized by being capable of softening natto with the progress of the fermentation. Also, it is intended to provide a method of producing soft natto by using the above-described strain and the soft natto produced by this production method.

### TECHNICAL FIELD

The present invention relates to a novel strain in natto-producing bacteria, and a novel natto produced by using said strain. More specifically, the invention relates to a novel strain in natto-producing bacteria with a potency to soften natto as fermentation progress and a soft natto produced by using said strain.

### BACKGROUND OF THE INVENTION

Natto is a traditional food produced by fermenting boiled soybean with natto-producing bacteria belonging to Bacillus subtilis. One of the factors influencing the quality of natto is hardness (or softness).

In the recent tendency of demanding soft natto, attempts have been made to produce soft natto, for example, by selecting and using soybean with a property of softness after boiling or by using boiled soybean with softness by enhancing the steaming intensity of soybean. However, disadvantageously, it has been difficult to stably obtain soybean with property of softness after boiling. Additionally, the color of steamed soybean has turned black in case that the steaming intensity of soybean is enhanced, disadvantageously, so that the quality of the resulting natto is deteriorated.

Meanwhile, it is reported that on comparison of the hardness of natto before fermentation with natto-producing bacteria with the hardness thereof after fermentation with natto-producing bacteria, the hardness of natto after fermentation is higher than that before fermentation (see, for example, non-patent reference 1). It is also reported that as fermentation progresses, additionally, the resulting natto is more hardened over time (see, for example, non-patent reference 2).

In such circumstances, it is expected to be able to stably produce soft natto which keeps other properties such as stickiness, umami-taste and color etc. without changing, when such a novel strain in natto-producing bacteria as being capable of softening natto with progress of fermentation and keeping other fermentation properties equal to those of the existing natto-producing bacteria can be obtained.

Actually no report exists, telling about the development of a strain in natto-producing bacteria with such properties.

Non-patent reference 1: Rep. Natl. Food Res. Inst., Vol. 51, p. 48-58, 1987
Non-patent reference 2: Nippon Shokuhin Kogyo Gakkaishi, Vol. 40, No. 1, p. 75-82, 1993

### DISCLOSURE OF THE INVENTION

### Object to be Solved by the Invention

The objects of the present invention are to provide a strain in natto-producing bacteria capable of softening natto as fermentation progresses, a method of producing soft natto by using the above-described strain, and the soft natto produced by this production method.

### Means for Solving the Object

So as to obtain a strain in natto-producing bacteria suitable for the production of soft natto, the present inventor made investigations in a wide variety of fields. The inventor could select the strain in natto-producing bacteria, not only being capable of softening natto as fermentation progresses but also keeping other fermentation properties equal to those of existing natto-producing bacteria, among isolates from chungkookjang. The chungkookjang is a traditional Korean fermented soybean food prepared by using rice straws, which is mainly ingested in Korea. Then, the inventor used the selected strain to produce natto, so that the inventor verified the production of soft natto using the selected strain. Thus, the invention has been achieved.

Specifically, the invention according to claim 1 relates to a strain in natto-producing bacteria capable of softening natto with the progress of the fermentation, which belongs to Bacillus subtilis.

Further, the invention according to claim 2 relates to a strain in natto-producing bacteria according to claim 1, characterized in that the 16s RNA gene thereof is consisting of the nucleotide sequence of SQ ID NO. 1 in the sequence listing.

Still further, the invention according to claim 3 relates to a strain in natto-producing bacteria according to claim 1 or 2, characterized in that the strain is Bacillus subtilis K3 (FERM BP-10801).

Additionally, the invention according to claim 4 relates to a method of producing soft natto, characterized in that the method comprises using a strain according to any one of claims 1 to 3.

Still additionally, the invention according to claim 5 relates to natto, characterized in that the natto is produced by a method of producing natto according to claim 4.

Still more additionally, the invention according to claim 6 relates to soft natto according to claim 5, characterized in that the soft natto is produced by a method of producing natto according to claim 4 and that the soft natto has a lower hardness compared with a hardness of the natto produced by using Bacillus subtilis OUV23481 (FERM BP-6659) as a control natto-producing strain.

Further, the invention according to claim 7 relates to soft natto according to claim 5, characterized in that the soft natto is produced by the method of producing soft natto according to claim 4 and in that the soft natto has a hardness of 0.2 to 0.4 N.

### ADVANTAGES OF THE INVENTION

In accordance with the invention, a strain in natto-producing bacteria capable of softening natto as fermentation progresses (the natto-producing strain capable of producing soft natto) is provided. Soft natto toward which the demand has been increasing in recent years can be produced by using the strain.

### BEST MODE FOR CARRYING OUT THE INVENTION

The invention is described in detail hereinbelow.

In accordance with the invention, strains in natto-producing bacteria were isolated.

Any source from which strains in natto-producing bacteria can be isolated may be used and includes for example but is not limited to rice straw, dead leaves, soil and fermented food products worldwide. For example, chungkookjang, that is a fermented soybean product commonly ingested in Korea is preferable.

Strains in natto-producing bacteria can be isolated as follows. Specifically, a sample as a source for isolating natto-producing bacteria is suspended in, for example, sterile water and is then diluted to an appropriate concentration; subsequently, the resulting diluted solution is plated on a standard agar medium (2.5 g of yeast extract, 5.0 g of trypton, 1 g of glucose, 15 g of agar per one liter, pH 7.1; manufactured by Eiken Chemical Co., Ltd.), and then for overnight culture at 37° C.; and the resulting colonies are isolated.

In this case, it is prefer to select colonies potentially considered as natto-producing bacteria by using the shapes of the colonies as a marker. Further, the selected colonies are applied on the SG agar medium (5% sucrose, 1.5% monosodium L-glutamate salt, 0.27% KH₂PO₄ (potassium dihydrogen phosphate), 0.42% Na₂HPO₄.12H₂O (disodium hydrogen phosphate·12 hydrates), 0.05% NaCl, 0.05% MgSa₄.7H₂O (magnesium sulfate·7 hydrates), 0.1 μg/ml biotin, 1.6% agar, pH 6.4; see for example JP-A-10-215861) and cultured for overnight at 37° C., to select colonies forming viscous materials.

A spore solution is prepared from the resulting each colony according to a general method (see for example JP-A-10-215861), which can be used as a seed preparation for producing natto.

Natto can be produced by a conventional method. For example, soybeans are soaked in water, then water is drained therefrom and then resulting soybeans are steamed at 0.18 MPa for 18 minutes; spores of 1000 to 4000 in number are inoculated per 1 g of the steamed soybean; each portion of 40 g is placed in each PSP tray; after the surface is coated with a thin film, the each tray is covered with a lid; and subsequently, the PSP trays are placed in a batch-type natto fermenter for fermentation at ambient temperature of 37 to 42° C. and high humidity. After completion of fermentation and aging, the quality of resulting natto is evaluated. Herein, the hardness of natto can be evaluated by a sensory test and by measuring a hardness (see for example JP-A-2004-24197).

The intended strains in natto-producing bacteria can be selected as follows: Specifically, natto is produced by using the each seed preparation from resulting colonies, so that strains in natto-producing bacteria having poor threading ability are excluded. Then, the strains in natto-producing bacteria capable of producing natto softer than natto produced by using an existing natto-producing bacteria, namely Bacillus subtilis OUV23481 (FERM BP-6659) (sometimes referred to as strain OUV23481 hereinafter) as a control (see for example JP-A-2000-287676) can be selected. Then, the quality of the natto produced by using the selected strains is evaluated, to select the strains in natto-producing bacteria excellent in terms of quality markers in addition to the hardness.

The Bacillus subtilis OUV23481 was deposited on Feb. 23, 1999 at the National Institute of Bioscience and Human-Technology, the Agency of Industrial Science and Technology, Ministry of International Trade and Industry (1-3, Higashi 1-chome Tsukuba-shi, Ibaraki-ken, Japan; zip code 305-8566) [currently under the name of the International Patent Organism Depositary, National Institute of the Advanced Industrial Science and Technology (AIST Tsukuba Central 6, 1-1, Higashi 1-chome, Tsukuba-shi, Ibaraki-ken, Japan; zip code 305-8566)]. The deposit No. is FERM BP-6659.

Any conventional method for utilizing the excellent strain in natto-producing bacteria capable of producing soft natto as selected in such manner may be used, with no specific limitation.

For example, natto is generally so-called whole soybean natto produced by using whole soybean as a raw material. Additionally, split natto from preliminarily split or cracked soybean as a raw material is also on market.

The general method for producing the whole soybean natto is as follows: Whole soybeans as a raw material are soaked in cold water for ten and several hours, subsequently are steamed at a pressure (0.15 to 0.20 MPa) using pressurized steam in a steam pan. Then, the seed preparation of natto-producing bacteria is inoculated to the resulting steamed soybeans and both are mixed at a state of high temperature (70 to 100° C.) Thereafter the mixture is placed in a conventional container and transferring the container in a fermenter for fermentation at ambient temperature of 37 to 42° C. for a general period of time (about 15 to 20 hours), and then aging the resulting natto around 5° C. under refrigeration (about 12 to 72 hours).

Furthermore, split natto is produced by the same method as for the general whole soybean natto, except for soaking preliminarily split soybean in water.

In accordance with the invention, the strain in natto-producing bacteria for use at the fermentation step by such conventional method for producing natto is replaced with the excellent natto-producing strain selected by the method described above to produce natto. In such manner, softer natto compared with conventional natto can be produced.

The natto produced by the method of the invention is soft immediately after production and is at a hardness (Newton: N) of about 50 to 75% of the hardness (N) of the natto produced by the control strain (the strain OUV23481) (in other words, the natto of the present invention is soft to that extent, compared with the control natto). The hardness (N) of the natto produced by the control strain increases with the progress of the fermentation time. On the contrary, the natto of the present invention is characterized by decreasing the hardness (N) with the progress of the fermentation.

In the 12th hour of fermentation, the hardness (N) of the natto of the present invention corresponds to 75 to 85% of the hardness (N) of the control natto, while in the 18th hour of fermentation, the hardness (N) of the natto of the present invention corresponds to 40 to 50% of the hardness (N) of the control natto. Thus, the hardness (N) of the natto of the present invention is lower than the hardness (N) of the control natto and is decreasing with the progress of the fermentation. As to the hardnesses (N) of the both natto, the hardness of the control natto in the 18th hour of fermentation increases to 110 to 135% of the hardness thereof in the 12th hour of fermentation. On the contrary, the hardness of the natto of the present invention in the 18th hour fermentation decreases to 55 to 75% (generally 60 to 70%) of the hardness thereof in the 12th hour of the fermentation.

In accordance with the present invention, therefore, softer natto compared with the control can be obtained; in addition, the hardness of the control natto is increasing with the progress of the fermentation, on the contrary, the natto of the present invention is softened, so that the present invention is characterized in that soft natto immediately after production can sufficiently be expected. As a matter of course, the natto of the present invention is verified to have the same properties of stickiness, hardness (by a sensory test) and umami-tasete, as those of the control natto. Thus, it can be said that the natto of the present invention is novel and is a quite useful food product.

### EXAMPLES

Examples of the invention are described hereinbelow.

### Example 1

### (1) Isolation of Natto-Producing Strain

An appropriate volume of chungkookjang was suspended in sterile water, diluted with sterile water, and then plated on a standard agar medium (manufactured by Eiken Chemical Co., Ltd.), and cultured for overnight at 37° C. The grown colonies were inoculated on the SG agar medium and cultured for 24 hours at 37° C., to select bacteria forming viscous materials. Spore solutions were prepared from the resulting bacteria to prepare natto by a conventional method.

(2) Evaluation of Natto

The stickiness of the produced natto was evaluated by a sensory test, to exclude natto types with a poor stickiness. Then, the hardness of natto was evaluated at a sensory test, to select soft natto. Further, the hardness of the selected natto was confirmed with a texture analyzer as follows:

Specifically, a texture analyzer equipped with a digital force gauge (type: FGC-0.2, manufactured by NIDEC-SHIMPO Corporation) on which a plunger having a diameter of 1.5 mm was mounted was used, while the digital force gauge was preliminarily set on a moterized test stand (type: FGS-50V-L, manufactured by NIDEC-SHIMPO Corporation) where the plunger element was downwardly set.

After natto to be measured was first warmed to 15 to 25° C., the natto was thoroughly loosened with chopsticks while avoiding the mashing of the natto bean; and then, appropriate one bean was placed on the table of the moterized test stand.

Subsequently, the digital force gauge was lowered at a speed of 60 mm/minute, to push and mash the bean while piercing the center of the bean with the plunger. Just when the plunger was lowered to 1±0.2 mm above the bottom of the bean, the lowering of the digital force gauge was stopped to record the indicated value (unit: Newton (N); the peak value).

After a total of 14 beans were measured to read the indicated values in the same manner as described above, the mean of the indicated values of 10 beans excluding the top two values and the bottom two values was calculated, to determine the hardness (N) of natto.

Under the same steaming conditions and fermentation conditions, natto was produced using the strain OUV23481 and thus produced natto was used as a control for natto evaluation.

Table 1 shows the stickiness of natto produced by using the selected strains in natto-producing bacteria by the method described above, the hardness (at the sensory test) of said natto and the hardness (N) thereof.

TABLE 1

| Name of bacterial | | | |
|---|---|---|---|
| strain | Stickiness | Hardness | Hardness (N) |
| Strain K3 | average | soft | 0.218 |
| Strain R1 | average | soft | 0.251 |
| Strain R4 | average | slightly soft | 0.325 |
| Strain W1 | average | soft | 0.276 |
| Strain W4 | slightly poor | slightly soft | 0.291 |
| Strain W5 | average | slightly soft | 0.297 |
| Strain OUV23481 | average | average | 0.431 |

Compared with the production by means of the strain OUV23481, softer natto could be produced when steamed soybean was fermented with these selected strains.

### Example 2

Natto was respectively produced by using the strain K3 by which the softest natto could be produced among the selected strains obtained in Example 1 and by using the strain OUV23481, according to the general method. The hardness of the resulting natto was measured with the progress of the fermentation since the initiation of fermentation. The results are shown in Table 2.

TABLE 2

| | Strain OUV 23481 | | | | | |
|---|---|---|---|---|---|---|
| Fer- | Hard- | | | Strain K3 | | |
| mentation | ness | | | Hardness | | |
| Period | (N) | stickiness | Umami | (N) | stickiness | Umami |
| 12 hours | 0.433 | poor | poor | 0.360 | poor | poor |
| 15 hours | 0.407 | slightly | poor | 0.326 | slightly | poor |
| | | poor | | | poor | |
| 18 hours | 0.555 | average | average | 0.246 | average | average |

In case of fermentation by using the existing Bacillus subtilis OUV23481 strain, the hardness at the final stage of fermentation increased. In other words, the resulting natto was hardened. Alternatively, natto fermented by using the strain K3 was softer than natto produced by using the strain OUV23481, throughout the time period of fermentation. The hardness of the natto produced by using the strain K3 decreased consistently with the progress of the fermentation during the fermentation period, so that the natto was softened (Table 2). With respect to all properties of umami-taste and stickiness, the natto produced by using the strain K3 was equal to the natto produced by using the strain OUV23481.

### Example 3

Table 3 shows the mycological properties of the strain K3 compared with Bacillus subtilis. Herein, the individual indexes were tested with reference to “The Bergey's Manual of Determinative Bacteriology, the 8^{th} edition” (The Williams and Wilkins Company, 1974), “Shokuhin Biseibutsugaku Handbook (Handbook for Food Bacteriology)” (issued by Giho-do Kabushiki Kaisha, 1995) and “Kokiseigaho keisei-kin no Zukan (The Illustrated Reference Book of Aerobic Spore-forming Bacteria)” (Japan Becton Dickinson Co., Ltd., 2004).

TABLE 3

| | | Bacillus |
|---|---|---|
| Index | Strain K3 | subtilis |
| Morphology | rod-shaped | rod-shaped |
| Gram staining | + | + |
| Spore formation | + | + |
| Growth in aerobic culture | + | + |
| Growth in anaerobic culture | − | − |
| Utilization of citrate | + | +/− |
| VP test | + | + |
| Formation of acid from glucose | + | + |
| Reduction of nitrate | − | +/− |
| Formation of acid from mannit | − | +/− |

Additionally, FIG. 1 and SQ ID NO. 1 in the sequence listing show the nucleotide sequence of the 16s RNA gene of the strain K3. The 16s RNA gene was amplified by using the primers shown in FIG. 2, to determine the nucleotide sequence.

Specifically, the nucleotide sequence of an amplified fragment obtained by PCR with GeneAmp PCR system 2400 (manufactured by Applied Biosystem Japan) by using as template the DNA of the strain K3, which is extracted with Quicklyse Miniprep Kits (manufactured by Qiagen), and the primers shown in FIG. 2 was determined with ABI PRISM 3700 (manufactured by Applied Biosystem Japan).

As the primers, 27F (SQ ID NO. 2), 520R (SQ ID NO. 3), 520F (SQ ID NO. 4), 920R (SQ ID NO. 5), 920F (SQ ID NO. 6) and 1492R (SQ ID NO. 7) were used. Herein, the nucleotide sequences of these primers are collectively shown in FIG. 2.

The homology of the nucleotide sequence of the 16sRNA gene of the strain K3 to that of Bacillus subtilis DSM10, which is the type strain of Bacillus subtilis, was 99.9% intensity or more.

Based on the aforementioned results, it was identified that the strain K3 was belonged to Bacillus subtilis, like the existing Bacillus natto strains.

As described above, the strain K3 is very characteristic from the standpoint of the production of soft natto. Therefore, the strain K3 was deposited as Bacillus subtilis strain K3 on Mar. 19, 2007 at the International Patent Organism Depositary, National Institute of Advanced Industrial Science and Technology (AIST Tsukuba Central 6, 1-1, Higashi 1-chome, Tsukuba-shi, Ibaraki-ken, Japan; zip code 305-8566). The deposit No. is FERM BP-10801.

Furthermore, the strain K3 readily turned sporogenic, unlike the Bacillus subtilis var. chungkookjang KCTC0697BP strain described in the publication of JP-A-2002-233391. Specifically, in any case of shaking culture in a nutrient medium (2% yeast extract, 0.5% NaCl, pH 7.0) at 37° C. for 48 hours and of shaking culture in a spore-forming culture medium (see for example JP-A-10-215861) at 37° C. for 24 hours it was confirmed that 90% or more of cells of the strain K3 has turned into spores. Hence, it was verified that the strain K3 was different from Bacillus subtilis var. chungkookjang.

### BRIEF DESCRIPTION OF THE DRAWINGS

FIG. 1 A view depicting the nucleotide sequence of 16sRNA of the strain K3.

FIG. 2 A view depicting the nucleotide sequences of the primers.

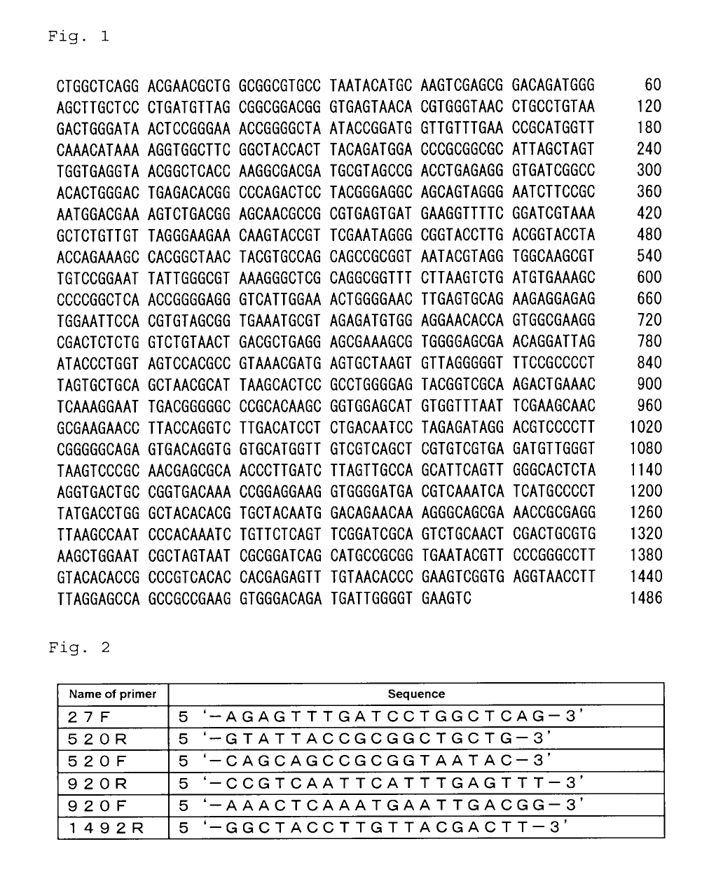

1. A strain in natto-producing bacteria capable of softening natto during the progress of the fermentation, said strain belonging to Bacillus subtilis.

2. The strain in natto-producing bacteria according to claim 1, wherein the 16s RNA gene thereof consists of the nucleotide sequence of SQ ID NO. 1 in the sequence listing.

3. The strain in natto-producing bacteria according to claim 1, wherein the strain is Bacillus subtilis K3 (FERM BP-10801).

4. A method of producing natto which comprises using a strain according to claim 1.

5. A soft natto, wherein the natto is produced by the method according to claim 4.

6. The soft natto according to claim 5, wherein the soft natto has a lower hardness compared with a hardness of a natto produced by using a Bacillus subtilis OUV23481 (FERM BP-6659) as a control natto-producing strain.

7. The soft natto according to claim 5, wherein the soft natto has a hardness of 0.2 to 0.4 N.


SEQUENCE LISTING (7 sequences)

SEQ ID NO: 1 - DNA, 1486 bp, Bacillus subtilis
ctggctcaggacgaacgctggcggcgtgcctaatacatgcaagtcgagcggacagatgggagcttgctccctgatgttagcggcggacgggtgagtaacacgtgggtaacctgcctgtaagactgggataactccgggaaaccggggctaataccggatggttgtttgaaccgcatggttcaaacataaaaggtggcttcggctaccacttacagatggacccgcggcgcattagctagttggtgaggtaacggctcaccaaggcgacgatgcgtagccgacctgagagggtgatcggccacactgggactgagacacggcccagactcctacgggaggcagcagtagggaatcttccgcaatggacgaaagtctgacggagcaacgccgcgtgagtgatgaaggttttcggatcgtaaagctctgttgttagggaagaacaagtaccgttcgaatagggcggtaccttgacggtacctaaccagaaagccacggctaactacgtgccagcagccgcggtaatacgtaggtggcaagcgttgtccggaattattgggcgtaaagggctcgcaggcggtttcttaagtctgatgtgaaagcccccggctcaaccggggagggtcattggaaactggggaacttgagtgcagaagaggagagtggaattccacgtgtagcggtgaaatgcgtagagatgtggaggaacaccagtggcgaaggcgactctctggtctgtaactgacgctgaggagcgaaagcgtggggagcgaacaggattagataccctggtagtccacgccgtaaacgatgagtgctaagtgttagggggtttccgccccttagtgctgcagctaacgcattaagcactccgcctggggagtacggtcgcaagactgaaactcaaaggaattgacgggggcccgcacaagcggtggagcatgtggtttaattcgaagcaacgcgaagaaccttaccaggtcttgacatcctctgacaatcctagagataggacgtccccttcgggggcagagtgacaggtggtgcatggttgtcgtcagctcgtgtcgtgagatgttgggttaagtcccgcaacgagcgcaacccttgatcttagttgccagcattcagttgggcactctaaggtgactgccggtgacaaaccggaggaaggtggggatgacgtcaaatcatcatgccccttatgacctgggctacacacgtgctacaatggacagaacaaagggcagcgaaaccgcgaggttaagccaatcccacaaatctgttctcagttcggatcgcagtctgcaactcgactgcgtgaagctggaatcgctagtaatcgcggatcagcatgccgcggtgaatacgttcccgggccttgtacacaccgcccgtcacaccacgagagtttgtaacacccgaagtcggtgaggtaaccttttaggagccagccgccgaaggtgggacagatgattggggtgaagtc

SEQ ID NO: 2 - DNA, 20 bp, Artificial Sequence
  Description of Artificial Sequence Synthetic primer
agagtttgatcctggctcag

SEQ ID NO: 3 - DNA, 18 bp, Artificial Sequence
  Description of Artificial Sequence Synthetic primer
gtattaccgcggctgctg

SEQ ID NO: 4 - DNA, 18 bp, Artificial Sequence
  Description of Artificial Sequence Synthetic primer
cagcagccgcggtaatac

SEQ ID NO: 5 - DNA, 20 bp, Artificial Sequence
  Description of Artificial Sequence Synthetic primer
ccgtcaattcatttgagttt

SEQ ID NO: 6 - DNA, 20 bp, Artificial Sequence
  Description of Artificial Sequence Synthetic primer
aaactcaaatgaattgacgg

SEQ ID NO: 7 - DNA, 19 bp, Artificial Sequence
  Description of Artificial Sequence Synthetic primer
ggctaccttgttacgactt